(19) United States
(12) Patent Application Publication
(10) Pub. No.: US 2014/0377347 A1
(43) Pub. Date: Dec. 25, 2014

(54) IN-SITU MULTILAYERED TABLET TECHNOLOGY

(71) Applicant: RANBAXY LABORATORIES LIMITED, New Delhi, Delhi (IN)

(72) Inventors: Kumaravel Vivek, Chennai (IN); Anuj Kumar Fanda, Ghaziabad (IN); Murali Krishna, Krishna (IN); Ravish Kumar Sharma, Khargone (IN); Kanwarpreet Singh Bakshi, Gurgaon (IN); Romi Barat Singh, Varanasi (IN); Ajay Kumar Singla, Gurgaon (IN)

(73) Assignee: RANBAXY LABORATORIES LIMITED, New Delhi, Delhi (IN)

(21) Appl. No.: 14/373,603

(22) PCT Filed: Jan. 23, 2013
(86) PCT No.: PCT/IB2013/050584

§ 371 Date: Jul. 21, 2014

(30) Foreign Application Priority Data
Jan. 23, 2012 (IN) 193/DEL/2012

(51) Int. Cl. A61K 9/24; A61K 31/426; A61K 31/196; A61K 31/4439; A61K 9/20; A61K 31/65

(52) U.S. Cl. CPC A61K 9/209; A61K 9/2086; A61K 31/65; A61K 31/196; A61K 31/4439; A61K 31/426; A61K 9/2031; A61K 9/2013; A61K 9/2009; A61K 9/2018; A61K 9/2054
USPC 424/465; 514/152; 514/567; 514/338; 514/370

(57) ABSTRACT

The present invention relates to an in-situ multilayered tablet comprising at least one polymer layers and at least one drug layers wherein the said layers are physically separated from each other. After coming in contact with biological and/or aqueous fluids at least one of the polymer layers rapidly swells and sticks to one or more drug layers to form an in-situ multilayered tablet. Further, the polymer layer may optionally comprise a drug. Furthermore, the present invention relates to the processes for preparing said in-situ multilayered tablets.

### FIELD OF THE INVENTION

The present invention relates to an in-situ multilayered tablet comprising at least one polymer layer and at least one drug layer, wherein said layers are physically separated from each other. After coming in to contact with biological and/or aqueous fluids, at least one of the polymer layers rapidly swells, and each such layer sticks to at least one drug layer to form an in-situ multilayered tablet. The polymer layer may also optionally comprise a drug. The present invention also relates to processes for preparing said in-situ multilayered tablets.

### BACKGROUND OF THE INVENTION

Oral drug delivery continues to be the most popular route of administration due to its versatility, ease of administration and probably most importantly patient compliance. An oral medication that improves compliance and thus results in more effective treatment has been one of the major drivers of innovation in the oral drug delivery market.

Controlled-release dosage form is an advancement in the oral drug delivery which has led to improved patient compliance and reduced side effects of the drugs. Controlled-release dosage form slows the release of the drug so that there is no need to take the drug too often and therefore improves compliance. The other benefit of controlled-release dosage forms is that the drug release is restrained and there are smaller peaks and troughs in blood levels thereby reducing the chance of peak effects and increasing the likelihood of therapeutic effectiveness for longer periods of time.

Generally, controlled-release systems can be categorized into two groups based on actions. Extended-release formulations deliver a portion of the total dose shortly after ingestion and the remainder over an extended time frame. Delayed-release systems provide steady dosing after passage through the stomach. Controlled drug delivery systems aim to maintain plasma concentration of drugs within the therapeutic window for a longer period of time, thereby to ensure sustained therapeutic action.

Further manipulation of delivery systems has led to the development of chronotherapeutic systems, where release enables a drug to take advantage of the natural biorhythms of the human body. Pulsatile drug delivery system provides a chronotherapeutic release to meet the needs of the patients suffering from diseases which follow the biological rhythm such as asthma, where the crises mostly happen late at night, osteoarthritis where the pain is again more intense during night, rheumatoid arthritis where the pain peaks at the morning; duodenal ulcer where the highest gastric secretion happens at night, neurological disorders such as epilepsy where the oscillations are follow melatonin secretion; hypercholesterolemia, where the cholesterol synthesis is higher during the night; and several cardiovascular diseases such as cardiac and/or platelet aggregation that majorly occur during early hours of the morning. Pulsatile drug delivery systems are characterized by at least two distinctive drug-release phases following a predetermined lag time. The drug's release may be controlled by time, by site, or a combination of the two parameters.

Two of the most widely commercialized controlled-release technologies are OROS® (developed by Alza), and the SODAS® technology developed by Elan Drug Technologies. Other successfully commercialized technologies include SkyePharma's Geomatrix™, Aptalis Pharma's Diffucaps® and Elan's CODAS®.

U.S. Pat. Nos. 5,318,558 and 5,221,278 claim the pulsatile delivery of agents from osmotic systems based on the technology of an expandable orifice.

U.S. Pat. No. 7,387,793 relates to a multi-particulate pharmaceutical dosage form wherein the active drug is layered onto a neutral core (such as cellulose spheres) and then one or more rate-controlling, functional membranes are applied.

U.S. Pat. No. 6,797,283 relates to a multilayered dosage form comprising: a first layer comprising an amount of swellable polymer, said amount being sufficient to swell said first layer such that the active agent dosage form is retained within the stomach of a subject; a second layer laminated with the first layer at a common surface, said second layer comprising a therapeutic amount of an active agent and being formulated to swell to a lesser extent than the first layer; and at least one band of insoluble material circumscribing only a portion of said first layer and said second layer, said at least one band of insoluble material binding together the first layer and the second layer.

U.S. Pat. No. 6,183,778 relates to an oral dosage form in the form of a tablet, capable of providing one or more pharmaceutically active substances in two or more different releases, the dosage form comprising at least three layers of specific geometric shape, wherein the first layer comprises an active ingredient and a substance which swells or solubilizes when contacted with aqueous liquids; the second layer is similar to the first layer but contains another active ingredient and the third layer partially coats one or more free surfaces of the second layer.

U.S. Pat. No. 5,783,212 discloses a multilayer tablet for the release of pharmaceutically active ingredient at a constant rate with a zero order kinetic profile, in which two outer layers contain swellable and erodible polymers, an inner layer contains a pharmaceutically active ingredient and swellable and erodible polymers, and each layer differs in composition and thickness.

U.S. Pat. No. 5,626,874 discloses a multilayer tablet consisting of two outer layers containing gellable or erodible polymers and an inner layer containing an active ingredient. The side surface of the inner layer occupies about 5% to 35% of the tablet's total surface.

U.S. Patent Application No. 2010/0040681 relates to an oral sustained-release triple layer tablet, more particularly, a triple layer tablet consisting of an inner immediate-release layer containing a pharmaceutically active ingredient and two outer layers containing swellable polymers. On exposure to aqueous media, the two outer layers swell to form gelled layers surrounding the lateral side of the inner layer rapidly, thereby effectively controlling the release of drug from the inner immediate-release layer.

U.S. Pat. No. 5,549,913 discloses a multilayered tablet for release of pharmaceutically active ingredient at a constant rate with a zero order kinetic profile, in which two outer layers contain pharmaceutically active ingredient and hydrophilic polymers, and an inner layer contains a water-soluble polymer without the pharmaceutically active ingredient. The inner layer is readily dissolved in aqueous media to separate the two outer layers, and thus to increase the surface area of the matrix.

U.S. Pat. No. 4,839,177 relates to a system for the controlled-rate release of active substances, consisting of a deposit-core comprising the active substance and having defined geometric form and a support-platform applied to said deposit-core. Said deposit-core contains, mixed with the active substance, a polymeric material having a high degree of swelling on contact with water or aqueous liquids, a gellable polymeric material, said polymeric materials being replaceable by a single polymeric material having both swelling and gelling properties, and other adjuvants able to provide the mixture with suitable characteristics for its compression and for its intake of water.

U.S. Pat. No. 5,780,057 relates to a two- or three-layered tablet, wherein at least one layer can rapidly swell by contact with biological and/or aqueous fluids, said swelling resulting in a considerable increase in the tablet volume. Said phenomenon determines a prolonged residence of the pharmaceutical form at the gastric level and therefore allows a slow-release of the active ingredient from said pharmaceutical form to the stomach and/or the first tract of the intestine.

The present inventors have developed a novel in-situ multilayered tablet comprising at least one polymer layer and at least one drug layer, wherein the said layers are physically separated from each other. After coming in contact with biological and/or aqueous fluids, at least one of the polymer layers rapidly swells and sticks to one or more drug layers to form an in-situ multilayered tablet. Further, the polymer layer may optionally comprise a drug. Furthermore, the in-situ multilayered tablet of the present invention provides an initial lag phase followed by the controlled-release of the drug present in the drug layer, wherein the drug layer is sandwiched between the two polymer layers. Initially, the drug release occurs from a limited area which is exposed; with time, the polymer layers erode and expose the drug layer completely. It may also provide an initial immediate-release followed by the controlled-release of the drug present in the drug layer, wherein the drug layer contains a polymer layer on either of its sides. Therefore, the present technology provides a controlled-release with an initial lag phase or an initial immediate-release. The present dosage form also provides the pulsatile release of the drug by the delivery of the drug from two or three different layers with different release rates. Further, the present dosage form can be used to formulate two or more incompatible drugs into a single dosage form.

### SUMMARY OF THE INVENTION

The present invention relates to an in-situ multilayered tablet.

One of the aspects of the present invention relates to an in-situ multilayered tablet comprising at least one polymer layer and at least one drug layer wherein the said layers are physically separated from each other.

According to one of the embodiments, the present invention relates to an in-situ bi-layered tablet comprising one polymer layer and one drug layer.

According to another embodiment, the present invention relates to an in-situ tri-layered tablet comprising two polymer layers and one drug layer.

According to another embodiment, the present invention relates to an in-situ four layered tablet comprising two polymer layers and two drug layers.

According to yet another embodiment of the present invention, the polymer layer may optionally comprise a drug.

According to one of the embodiments of the present invention, the polymer layer and the drug layer contain the same drug.

According to another embodiment of the present invention, the polymer layer and the drug layer contain different drugs.

According to another aspect, the in-situ multilayered tablet of the present invention provides an initial lag phase followed by the controlled-release of the drug present in the drug layer, wherein the drug layer is sandwiched between the two polymer layers.

According to another aspect, the in-situ multilayered tablet of the present invention provides an initial immediate-release followed by the controlled-release of the drug present in the drug layer, wherein the drug layer contains a polymer layer on either of its sides.

According to another aspect, the in-situ multilayered tablet of the present invention comprises an immediate-release layer on it.

According to one of the embodiments the immediate-release layer is in the form of a powder or a tablet.

According to another aspect, the polymer layer comprises swelling polymers, antiadherents, binders, diluents, disintegrants, glidants, lubricants, opaquants and/or polishing agents and optionally a drug.

According to one of the embodiments, the swelling polymer is selected from the group consisting of polyethylene oxide polymers, polyethylene glycol polymers, hydroxymethylcellulose, hydroxyethylcellulose, hydroxypropyl methylcellulose having molecular weight from 1,000 to 4,000,000, hydroxypropyl cellulose having molecular weight from 2,000 to 2,000,000, carboxyvinyl polymers, polyvinyl alcohols, glucans, scleroglucans, chitosans, mannans, galactomannans, xanthan gum, carrageenan, amylose, alginic acid and salts and derivatives thereof, polyanhydrides, polyamino acids, methyl vinyl ethers/maleic anhydride copolymers, carboxymethylcellulose and derivatives thereof, acrylates, methacrylates, acrylic/methacrylic copolymers, or mixtures thereof.

According to another embodiment, the swelling polymer comprises about 50% to about 100% by weight of the polymer layer.

According to another aspect, the drug layer comprises a drug and one or more pharmaceutically acceptable excipients selected from the group comprising adsorbents, antioxidants, acidifying agents, alkalizing agents, buffering agents, colorants, flavorants, sweetening agents, antiadherents, binders, diluents, disintegrants, glidants, lubricants, opaquants and/or polishing agents.

According to another aspect, the polymer layer and the drug layer are prepared by the process of direct compression, dry granulation or wet granulation.

### DESCRIPTION OF THE INVENTION

The present invention relates to a novel in-situ multilayered tablet.

The phrase “in-situ multilayered tablet”, as used herein, relates to a tablet having two or more physically separated layers outside the body wherein after coming in contact with biological and/or aqueous fluids at least one of the layers rapidly swell and stick to the other layers to form an in-situ multilayered tablet.

The term “drug”, as used herein, relates to any therapeutic or diagnostic agent now known or hereinafter discovered that can be formulated as described herein. It may be selected from the group consisting of pharmaceutically acceptable compounds including analgesics, antacids, anticonvulsants, anesthetics, antidiabetic agents, antibiotics, anti-acne agents anti-infective agents, antineoplastics, antiparkinsonian agents, antirheumatic agents, cardiovascular agents, central nervous system stimulants, dopamine receptor agonists, gastrointestinal agents, psychotherapeutic agents, or urinary tract agents.

Suitable examples of drugs which can be incorporated into the dosage form of the present invention include, but are not limited to, albuterol sulfate, amoxicillin, bupropion hydrochloride, carbidopa, cefaclor, diclofenac sodium, erythromycin, felodipine, loratidine, lithium carbonate, methylphenidate, metoprolol tartrate, nifedipine, propranolol, verapamil hydrochloride, omeprazole, esomeprazole, famotidine, sotalol hydrochloride, theophylline, terbutaline sulphate, enalapril, diltiazem, nifedipine, lovastatin, simvastatin, ibuprofen, indomethacin, tenoxicam, acetylsalicylic acid, and minocycline hydrochloride.

The phrase “pharmaceutically acceptable excipient”, as used herein, denotes any material which is inert in the sense that it substantially does not have any therapeutic and/or prophylactic effect per se. Such an excipient may be added with the purpose of making it possible to obtain a pharmaceutical composition which has acceptable technical properties.

As used herein, the term “alkalizing agent” is intended to mean a compound used to provide an alkaline medium for product stability.

As used herein, the term “acidifying agent” is intended to mean a compound used to provide an acidic medium for product stability.

The term “about”, as used herein, means up to plus or minus 10% of the particular term.

The in-situ multilayered tablet of the present invention comprises at least one polymer layer and at least one drug layer, wherein the said layers are physically separated from each other. After coming in contact with biological and/or aqueous fluids, at least one of the polymer layers rapidly swells and sticks to one or more drug layers to form an in-situ multilayered tablet. Further, the polymer layer may optionally comprise a drug. Furthermore, the in-situ multilayered tablet of the present invention provides an initial lag phase followed by the controlled-release of the drug present in the drug layer, wherein the drug layer is sandwiched between two polymer layers. Initially the drug-release occurs from a limited area which is exposed, with time the polymer layers erode and expose the drug layer completely. It may also provide an initial immediate-release followed by the controlled-release of drug present in the drug layer, wherein the drug layer contains polymer layer on either of its sides. Therefore, the present technology provides a controlled-release with an initial lag phase or an initial immediate-release. The present dosage form also provides the pulsatile-release of the drug by the delivery of the drug from two or three different layers with different release rates. Further, the present dosage form can be used to formulate two or more incompatible drugs into a single dosage form. Furthermore, the polymer layer or the drug layer of the present invention may be in the form of pre-compressed powder or a tablet, particularly in the form of tablets, wherein said tablets are filled in a capsule in a sequential manner.

The polymer layer comprises swelling polymers, antiadherents, binders, diluents, disintegrants, glidants, lubricants opaquants and/or polishing agents and optionally a drug.

The polymer layer comprises drug and polymer in a ratio of 0.0 to 2.0. Particularly the drug and polymer are present in a ratio of 0.1 to 0.5.

The drug layer comprises a drug and one or more pharmaceutically acceptable excipients selected from the group comprising extended-release polymer, delayed-release polymer adsorbents, antioxidants, acidifying agents, alkalizing agents, buffering agents, colorants, flavorants, sweetening agents, antiadherents, binders, diluents, disintegrants, glidants, lubricants, opaquants and/or polishing agents.

The drug layer comprises drug and polymer in a ratio of 0.01 to 2.0. Particularly, the drug and polymer are present in a ratio of 0.1 to 1.0.

Suitable examples of swelling polymers include polyethylene oxide polymers, polyethylene glycol polymers, hydroxymethylcellulose, hydroxyethylcellulose, hydroxypropyl methylcellulose having molecular weight from 1,000 to 4,000,000, hydroxypropyl cellulose having molecular weight from 2,000 to 2,000,000, carboxyvinyl polymers, polyvinyl alcohols, glucans, scleroglucans, chitosans, mannans, galactomannans, xanthan gums, carrageenan, amylose, alginic acid and salts and derivatives thereof, polyanhydrides, polyamino acids, methyl vinyl ethers/maleic anhydride copolymers, carboxymethylcellulose and derivatives thereof, acrylates, methacrylates, acrylic/methacrylic copolymers, or mixtures thereof.

The swelling polymer comprises about 50% to about 100% by weight of the polymer layer.

Suitable examples of binders include, but are not limited to, polyvinylpyrrolidone, starch mucilage, pregelatinized starch, sodium alginate, alginic acid, acacia mucilage, tragacanth, hydroxypropylmethyl cellulose, carboxymethylcellulose sodium, carboxymethylcellulose calcium, microcrystalline cellulose, ethyl cellulose, polyethylene glycol, hydroxyethyl cellulose, hydroxypropyl cellulose, methyl cellulose, polymethacrylates, carboxyvinyl polymers, carbopols, or mixtures thereof.

Suitable examples of diluents include, but are not limited to, corn starch, lactose, white sugar, sucrose, sugar-compressible, sugar confectioners, glucose, sorbitol, calcium carbonate, calcium phosphate-dibasic, calcium phosphate-tribasic, calcium sulfate, microcrystalline cellulose, silicified microcrystalline cellulose, cellulose powdered, dextrates, dextrins, dextrose, fructose, kaolin, lactitol, mannitol, starch, pregelatinized starch, or mixtures thereof.

Suitable examples of disintegrants include, but are not limited to, cross-linked polyvinylpyrrolidone, sodium starch glycolate, cross-linked sodium carboxymethyl cellulose (crosscarmellose sodium), calcium carboxymethyl cellulose, alginic acid and alginates, pregelatinised starch, starch and starch derivatives, low-substituted hydroxypropyl cellulose, or mixtures thereof.

Examples of lubricants and glidants include, but are not limited to, colloidal anhydrous silica, stearic acid, magnesium stearate, calcium stearate, talc, hydrogenated castor oil, sucrose esters of fatty acids, microcrystalline wax, yellow beeswax, white beeswax, or mixtures thereof.

Examples of antioxidants include, but are not limited to, ascorbic acid, ascorbyl palmitate, butylated hydroxyanisole, butylated hydroxytoluene, hypophosphorous acid, monothioglycerol, propyl gallate, sodium ascorbate, sodium bisulfite, sodium formaldehyde sulfoxylate, sodium metabisulfite, or mixtures thereof.

Suitable examples of alkalizing agents include, but are not limited to, ammonia solution, ammonium carbonate, diethanolamine, monoethanolamine, potassium hydroxide, sodium borate, sodium carbonate, sodium bicarbonate, sodium hydroxide, triethanolamine, diethanolamine, organic amine base, alkaline amino acids, trolamine, or mixtures thereof.

Suitable examples of acidifying agents include, but are not limited to, acetic acid, acidic amino acids, citric acid, fumaric acid and other alpha hydroxy acids, hydrochloric acid, ascorbic acid, phosphoric acid, sulfuric acid, tartaric acid, nitric acid, or mixtures thereof.

Examples of plasticizers include, but not limited to, triethyl citrate, tributyl citrate, triacetin, polyethylene glycol, propylene glycol, diethylphthatate, oils/glycerides such as fractionated coconut oil or castor oil, and any combination thereof.

Coloring agents and flavoring agents may be selected from any FDA approved colors and flavors for oral use.

Suitable extended-release polymers may be selected from one or more of water-miscible polymers, water-insoluble polymers, oils and oily materials, or mixtures thereof.

The water-miscible polymer may be selected from one or more of hydroxypropyl methylcellulose, hydroxypropyl cellulose, methylcellulose, sodium carboxymethylcellulose, hydroxyethyl cellulose and other cellulose derivatives, polymethacrylic copolymer, poloxamers, polyoxyethylene stearate, polyvinylpyrrolidone, polyvinylpyrrolidone-polyvinylacetate copolymer (PVP-PVA), polyvinyl alcohol, polyethylene oxide, or mixtures thereof. Particularly, the water-insoluble polymer may be selected from one or more of ethyl cellulose, cellulose acetate, cellulose nitrate, and mixtures thereof.

The oil or oily material may be hydrophilic, hydrophobic or oily material or their mixtures. Hydrophilic oil or oily material may be polyether glycols such as polypropylene glycols; polyoxyethylenes; polyoxypropylenes; poloxamers; polyglycolized glycerides such as gelucire, or mixtures thereof. Hydrophobic oil or oily material may be straight chain saturated hydrocarbons; sorbitan esters such as sorbitan diisostearate, or sorbitan dioleate, sorbitan monolaurate, sorbitan monoisostearate, sorbitan monooleate, sorbitan monopalmitate, sorbitan monostearate, sorbitan sesqui-isostearate, sorbitan sesquioleate, sorbitan sesquistearate, sorbitan tri-isostearate, sorbitan trioleate, sorbitan tristearate; higher fatty acid such as stearic acid, myristic acid, palmitic acid; higher alcohols such as cetanol or stearyl alcohol; waxes such as glyceryl monostearate, glyceryl monooleate, hydrogenated tallow, myristyl alcohol, stearyl alcohol, yellow beeswax, white beeswax, carnauba wax, castor wax, or substituted and/or unsubstituted mono, di or triglycerides; NVP polymers; PVP polymers; acrylic polymers, or mixtures thereof.

Suitable examples of delayed-release polymers include, but are not limited to, cellulose acetate phthalate (CAP), cellulose acetate trimellitate (CAT), poly(vinyl acetate)phthalate (PVAP), hydroxypropyl methylcellulose phthalate (HPMCP), poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, Eudragit® L-30-D (MA-EA, 1:1), Eudragit® L-100-55 (MA-EA, 1:1), Eudragit® L100, Eudragit® L12,5, Eudragit® S100, Eudragit® S12,5), Poly(methyl acrylate-co-methyl methacrylate-co-methacrylic acid) 7:3:1-(Eudragit® FS30D) hydroxypropyl methylcellulose acetate succinate, COATERIC™ (PVAP), AQUATERIC® CAP), AQOAT® (HPMCAS), or combinations thereof.

The polymer layer and/or the drug layer of the present invention may optionally contain surfactants.

Surfactants include both non-ionic and ionic (cationic, anionic and zwitterionic) surfactants suitable for use in pharmaceutical compositions. These include, but are not limited to, polyethoxylated fatty acid esters, polyethylene glycol fatty acid esters, alcohol-oil transesterification products, polyglycerized fatty acids, polyethylene glycol sorbitan fatty acid esters, sugar esters, polyoxyethylene-polyoxypropylene block copolymers, ionic surfactants, derivatives of fat soluble vitamins, and mixtures thereof. Suitable examples include sodium lauryl sulphate, sodium dodecyl sulphate, polyoxyethylene castor oil derivatives, for example, tweens, polyoxyethylene-polyoxypropylene block copolymers, for example, poloxamer, or mixtures thereof.

The polymer layer or the drug layer of the present invention is prepared by direct compression, dry granulation, wet granulation, or any other process known in the art.

The following examples represent various embodiments according to the present invention. The examples are given solely for the purpose of illustration and are not to be construed as limitations of the present invention, as many variations thereof are possible without departing from the spirit and scope of the invention.

### EXAMPLES

### Example 1

### Polymer Layer

| | | Percent (%) |
|---|---|---|
| S. No. | Ingredients | weight by weight |
| 1 | Polyethylene oxide | 98.0 |
| 2 | Magnesium Stearate | 1.3 |
| 3 | Colloidal silicon dioxide | 0.7 |

### Drug Layer

| | | Percent (%) |
|---|---|---|
| | | weight |
| S.No. | Ingredients | by weight |
| 1 | Minocycline hydrochloride | 32.4 |
| 2 | Lactose monohydrate | 47.6 |
| 3 | Hydroxypropylmethyl | 12.0 |
| | cellulose (HPMC E50) | |
| 4 | Hydroxypropylmethyl | 6.0 |
| | cellulose (HPMC E4MCR) | |
| 5 | Magnesium stearate | 1.0 |
| 6 | Colloidal silicon dioxide | 1.0 |

### Manufacturing Process:

### Polymer Layer

1. Polyethylene oxide was passed through the screen and lubricated using magnesium stearate and colloidal silicon dioxide.
2. The blend of step 1 was compressed into tablets.

### Drug Layer

1. Ingredients 1-4 of the drug layer were blended together.
2. The blend of step 1 was lubricated using magnesium stearate and colloidal silicon dioxide.
3. Finally, the blend of step 2 was compressed into tablets

### Filling of Tablets in to the Capsule

The tablets were filled in to the capsule in the following order:
1. Polymer tablet
2. Drug core tablet
3. Polymer tablet

### Example 2a

### Polymer Layer without Diluent (Polyethylene Oxide as the Gelling Polymer)

### Polymer Layer

| | | Percent (%) |
|---|---|---|
| | | weight |
| S. No. | Ingredients | by weight |
| 1 | Polyethylene oxide | 99.0 |
| 2 | Magnesium Stearate | 0.5 |
| 3 | Colloidal silicon dioxide | 0.5 |

### Drug Layer

| | | Percent (%) |
|---|---|---|
| S. No. | Ingredients | weight by weight |
| 1 | Diclofenac sodium | 10.0 |
| 2 | Lactose monohydrate | 30.0 |
| 3 | Hydroxypropylmethyl cellulose | 30.0 |
| 4 | Methacrylic Acid - Ethyl | 23.0 |
| | Acrylate Copolymer (1:1) Type A | |
| 5 | Polyvinyl pyrrolidine | 5.0 |
| 6 | Isopropyl alcohol | q.s |
| 7 | Magnesium stearate | 1.0 |
| 8 | Colloidal silicon dioxide | 1.0 |

### Manufacturing Process:

### Polymer Layer

1. Pass polyethylene oxide through a screen and lubricate using magnesium stearate and colloidal silicon dioxide.
2. Compress the blend of step 1 into tablets.

### Drug Layer

1. Weigh accurately ingredients 1-4 of the drug layer and pass through mesh.
2. Make a binder solution by dissolving polyvinyl pyrrolidine in isopropyl alcohol.
3. Granulate the powder mass of step 1 in a rapid mixer granulator using the binder solution of step 2.
4. Dry the granules of step 3 in a fluidized bed drier.
5. Mill the granules of step 4 and lubricate them using magnesium stearate and colloidal silicon dioxide.
6. Compress the granules of step 5 into tablets.

### Filling of Tablets in to a Hydroxypropylmethyl Cellulose Capsule

Fill the tablets in to a capsule in the following order:
1. Polymer tablet
2. Drug core tablet
3. Polymer tablet

### Example 2b

### Polymer Layer without Diluent (Polyethylene Oxide as the Gelling Polymer)

### Polymer Layer

| | | Percent (%) |
|---|---|---|
| S. No. | Ingredients | weight by weight |
| 1 | Polyethylene oxide | 98.0 |
| 2 | Magnesium stearate | 1.0 |
| 3 | Colloidal silicon dioxide | 1.0 |

### Drug Layer

| | | Percent (%) |
|---|---|---|
| S. No. | Ingredients | weight by weight |
| 1 | Diclofenac sodium | 20.0 |
| 2 | Mannitol | 20.0 |
| 3 | Hydroxypropylmethyl cellulose | 20.0 |
| 4 | Hydroxypropylmethyl cellulose phthalate | 35.0 |
| 5 | Hydroxypropyl cellulose-L | 4.0 |
| 6 | Isopropyl alcohol + water | q.s. |
| 7 | Magnesium stearate | 0.5 |
| 8 | Colloidal silicon dioxide | 0.5 |

### Manufacturing Process:

### Polymer Layer

1. Pass polyethylene oxide through a screen and lubricate using magnesium stearate and colloidal silicon dioxide.
2. Compress the blend of step 1 into tablets.

### Drug Layer

1. Weigh accurately ingredients 1-4 of the drug layer and pass through a mesh.
2. Make a binder solution by dissolving hydroxyl propyl cellulose-L in the mixture of isopropyl alcohol and water.
3. Granulate the powder mass of step 1 with the binder solution of step 2.
4. Dry the granules of step 3 in a fluidized bed drier.
5. Mill the granules of step 4 and lubricate them using magnesium stearate and colloidal silicon dioxide.
6. Compress the granules of step 5 into tablets.

### Filling of Tablets in a Hydroxypropylmethyl Cellulose Capsule

Fill the tablets in a capsule in the following order:
1. Polymer tablet
2. Drug core tablet
3. Polymer tablet

### Example 3a

### Polymer Layer with Diluent (Polyethylene Oxide as the Gelling Polymer)

### Polymer Layer

| | | Percent (%) |
|---|---|---|
| S. No. | Ingredients | weight by weight |
| 1 | Polyethylene oxide | 75.0 |
| 2 | Lactose monohydrate | 23.0 |
| 3 | Magnesium stearate | 1.0 |
| 4 | Colloidal silicon dioxide | 1.0 |

### Drug Layer

| | | Percent (%) |
|---|---|---|
| S. No. | Ingredients | weight by weight |
| 1 | Diclofenac sodium | 25.0 |
| 2 | Lactose anhydrous | 20.0 |
| 3 | Hydroxypropylmethyl cellulose | 20.0 |
| 4 | Methacrylic Acid - Ethyl Acrylate | 30.0 |
| | Copolymer (1:1) Type A | |
| 5 | Polyvinyl pyrrolidine | 4.0 |
| 6 | Isopropyl alcohol (IPA) + water | q.s. |
| 7 | Magnesium stearate | 0.5 |
| 8 | Colloidal silicon dioxide | 0.5 |

### Manufacturing Process:

### Polymer Layer

1. Pass polyethylene oxide and lactose monohydrate through screen and lubricate using magnesium stearate and colloidal silicon dioxide.
2. Compress the blend of step 1 into tablets.

### Drug Layer

1. Weigh accurately ingredients 1-4 of the drug layer and pass through a mesh.
2. Make a binder solution by dissolving polyvinyl pyrrolidine in the mixture of isopropyl alcohol and water.
3. Granulate the powder mass of step 1 with the binder solution of step 2.
4. Dry the granules of step 3 in a fluidized bed drier.
5. Mill the granules of step 4 and lubricate them using magnesium stearate and colloidal silicon dioxide.
6. Compress the granules of step 5 into tablets.

### Filling of Tablets in to a Hydroxypropylmethyl Cellulose/Hard Gelatin Capsule

Fill the tablets in a capsule in the following order:
1. Drug core tablet
2. Polymer tablet
3. Drug core tablet

### Example 3b

### Polymer Layer with Diluent (Polyethylene Oxide as the Gelling Polymer)

### Polymer Layer

| | | Percent (%) |
|---|---|---|
| S. No. | Ingredients | weight by weight |
| 1 | Polyethylene oxide | 80.0 |
| 2 | Mannitol | 18.0 |
| 3 | Magnesium stearate | 1.0 |
| 4 | Colloidal silicon dioxide | 1.0 |

### Drug Layer

| | | Percent (%) |
|---|---|---|
| S. No. | Ingredients | weight by weight |
| 1 | Diclofenac sodium | 30.0 |
| 2 | Lactose monohydrate | 20.0 |
| 3 | Hydroxypropylmethyl cellulose | 20.0 |
| 4 | Hydroxypropylmethyl cellulose phthalate | 24.0 |
| 5 | Polyvinyl pyrrolidine | 5.0 |
| 6 | Isopropyl alcohol + Water | q.s |
| 7 | Magnesium stearate | 0.5 |
| 8 | Colloidal silicon dioxide | 0.5 |

### Manufacturing Process:

### Polymer Layer

1. Pass polyethylene oxide and mannitol through a screen and lubricate using magnesium stearate and colloidal silicon dioxide.
2. Compress the blend of step 1 into tablets.

### Drug Layer

1. Accurately weigh ingredients 1-4 of the drug layer and pass through a mesh.
2. Make a binder solution by dissolving polyvinyl pyrrolidine in the mixture of isopropyl alcohol and water.
3. Granulate the powder mass of step 1 with the binder solution of step 2.
4. Dry the granules of step 3 in a fluidized bed drier.
5. Mill the granules of step 4 and lubricate them using magnesium stearate and colloidal silicon dioxide.
6. Compress the granules of step 5 into tablets.

### Filling of Tablets in a Hydroxypropylmethyl Cellulose/Hard Gelatin Capsule

Fill the tablets in a capsule in the following order:
Drug core tablet
Polymer tablet
Drug core tablet

### Example 4

### Hydroxypropylmethyl Cellulose as the Gelling Polymer in Polymer Layer and Polyethylene Oxide in Drug Layer

### Polymer Layer

| | | Percent (%) |
|---|---|---|
| S. No. | Ingredients | weight by weight |
| 1 | Hydroxy propylmethyl cellulose | 90.0 |
| 2 | Mannitol | 9.0 |
| 3 | Magnesium stearate | 0.5 |
| 4 | Colloidal silicon dioxide | 0.5 |

### Drug Layer

| | | Percent (%) |
|---|---|---|
| S. No. | Ingredients | weight by weight |
| 1 | Diclofenac sodium | 25.0 |
| 2 | Lactose monohydrate | 25.0 |
| 3 | Polyethylene oxide | 25.0 |
| 4 | Methacrylic Acid - Ethyl Acrylate | 20.0 |
| | Copolymer (1:1) Type A | |
| 5 | Hydroxypropyl cellulose-L | 4.0 |
| 6 | Isopropyl alcohol (IPA) + water | q.s. |
| 7 | Magnesium stearate | 0.5 |
| 8 | Colloidal silicon dioxide | 0.5 |

### Manufacturing Process:

### Polymer Layer

1. Pass hydroxypropylmethyl cellulose and mannitol through a screen and lubricate using magnesium stearate and colloidal silicon dioxide.
2. Compress the blend of step 1 into tablets.

### Drug Layer

1. Accurately weigh ingredients 1-4 of the drug layer and pass through a mesh.
2. Make a binder solution by dissolving hydroxypropyl cellulose-L in the mixture of isopropyl alcohol and water.
3. Granulate the powder mass of step 1 with the binder solution of step 2.
4. Dry the granules of step 3 in a fluidized bed drier.
5. Mill the granules of step 4 and lubricate them using magnesium stearate and colloidal silicon dioxide.
6. Compress the granules of step 5 into tablets.

### Filling of Tablets in to a Hydroxypropylmethyl Cellulose Capsule

Fill the tablets in a capsule in the following order:
1. Drug core tablet
2. Drug core tablet
3. Polymer tablet

### Example 5

### For Combination of Different Drugs: Pellet+ In Situ Trilayer Forming Tablet

### Omeprazole DR Pellets

| | | |
|---|---|---|
| | | Percent (%) |
| S. No. | Ingredients | weight by weight |

Core Beads

| | | |
|---|---|---|
| 1 | Omeprazole base | 13.25 |
| 2 | Mannitol | 49.67 |
| 3 | Microcrystalline cellulose | 9.93 |
| 4 | Sodium carbonate | 0.66 |
| 5 | Sodium lauryl sulphate | 0.33 |
| 6 | Hydroxypropyl cellulose-L | 0.66 |
| 7 | Purified water | q.s. |

Seal Coating

| | | |
|---|---|---|
| 8 | Hydroxypropyl methyl cellulose | 8.28 |
| 9 | Purified water | q.s. |

Enteric Coating

| | | |
|---|---|---|
| 10 | Methacrylic acid copolymer | 14.90 |
| 11 | Polyethylene glycol | 1.66 |
| 12 | Triethyl citrate | 0.33 |
| 13 | Purified water | q.s. |

Lubrication

| | | |
|---|---|---|
| 14 | Magnesium stearate | 0.33 |

### Polymer Layer

| | | Percent (%) |
|---|---|---|
| S. No. | Ingredients | weight by weight |
| 1 | Polyethylene oxide | 80.0 |
| 2 | Lactose monohydrate | 19.0 |
| 3 | Magnesium stearate | 0.5 |
| 4 | Colloidal silicon dioxide | 0.5 |

### Drug Layer

| | | Percent (%) |
|---|---|---|
| S. No. | Ingredients | weight by weight |
| 1 | Diclofenac sodium | 25.0 |
| 2 | Lactose monohydrate | 20.0 |
| 3 | Hydroxypropylmethyl cellulose | 20.0 |
| 4 | Methacrylic acid - ethyl acrylate | 30.0 |
| | copolymer (1:1) Type A | |
| 5 | Hydroxypropyl cellulose-L | 4.0 |
| 6 | Isopropyl alcohol (IPA) + water | q.s. |
| 7 | Magnesium stearate | 0.5 |
| 8 | Colloidal silicon dioxide | 0.5 |

### Manufacturing Process:

### Omeprazole DR Pellets

1. Dry mix ingredients 1-5 in a Rapid Mixer Granulator.
2. Granulate the powder mixture of step 1 with a solution of hydroxypropyl cellulose-L in water.
3. Add additional purified water to prepare a wet mass suitable for extrusion/spheronization.
4. Extrude the wet mass through an extruder.
5. Spheronize the wet extrudes using a spheronizer.
6. Dry the wet spheres.
7. Seal coat the dry spheres using an aqueous solution of hydroxypropylmethyl cellulose.
8. Enteric coat the seal coated pellets with an aqueous methacrylic acid copolymer dispersion.
9. Lubricate with magnesium stearate.

### Polymer Layer

1. Pass polyethylene oxide and lactose monohydrate through screen and lubricate using magnesium stearate and colloidal silicon dioxide.
2. Compress the blend of step 1 into tablets.

### Drug Layer

1. Accurately weigh ingredients 1-4 of the drug layer and pass through a mesh.
2. Make a binder solution by dissolving hydroxypropyl cellulose-L in the mixture of isopropyl alcohol and water.
3. Granulate the powder mass of step 1 with the binder solution of step 2.
4. Dry the granules of step 3 in a fluidized bed drier.
5. Mill the granules of step 4 and lubricate them using magnesium stearate and colloidal silicon dioxide.
6. Compress the granules of step 5 into tablets.

### Filling of Tablets and Pellets in to a Hydroxypropylmethyl Cellulose Capsule

Fill the tablets in a capsule in the following order:
1. Omeprazole pellets
2. Polymer tablet
3. Drug core tablet
4. Polymer tablet

### Example 6

### For Combination of Different Drugs

Polymer Layer with Drug 1 (Famotidine)

| | | Percent (%) |
|---|---|---|
| S. No. | Ingredients | weight by weight |
| 1 | Famotidine | 20.0 |
| 2 | Polyethylene oxide | 70.0 |
| 3 | Lactose anhydrous | 9.0 |
| 4 | Magnesium stearate | 0.5 |
| 5 | Colloidal silicon oxide | 0.5 |

### Drug 2 (Diclofenac) Layer

| | | Percent (%) |
|---|---|---|
| S. No. | Ingredients | weight by weight |
| 1 | Diclofenac sodium | 20.0 |
| 2 | Lactose anhydrous | 26.0 |
| 3 | Hydroxypropylmethyl cellulose | 25.0 |
| 4 | Hydroxypropylmethyl cellulose phthalate | 25.0 |
| 5 | Hydroxypropyl cellulose-L | 2.0 |
| 6 | Isopropyl alcohol (IPA) + Water | q.s |
| 7 | Magnesium stearate | 1.0 |
| 8 | Colloidal silicon dioxide | 1.0 |

### Manufacturing Process:

### Polymer Layer With Drug 1

1. Pass ingredients 1-3 through a sieve and lubricate using magnesium stearate and colloidal silicon dioxide.
2. Compress the blend of step 1 into tablets.

### Drug Layer

1. Accurately weigh ingredients 1-4 of the drug layer and pass through a mesh.
2. Make a binder solution by dissolving hydroxypropyl cellulose-L in the mixture of isopropyl alcohol and water.
3. Granulate the powder mass of step 1 with binder solution of step 2.
4. Dry the granules of step 3 in a fluidized bed drier.
5. Mill the granules of step 4 and lubricate them using magnesium stearate and colloidal silicon dioxide.
6. Compress the granules of step 5 into tablets.

### Filling of Tablets and Pellets in a Hydroxypropylmethyl Cellulose Capsule

Fill the tablets in a capsule in the following order:
1. Polymer tablet with drug 1
2. Drug 2 core tablet
3. Polymer tablet with drug 1

### Example 7

### For Combination of Different Drugs

### Famotidine Drug-Layering Dispersion

| | | Percent (%) |
|---|---|---|
| S. No. | Ingredients | weight by weight |
| 1 | Famotidine | 60.0 |
| 2 | Hydroxypropylmethyl cellulose | 40.0 |
| 3 | Water | q.s. |

### Polymer Layer

| | | Percent (%) |
|---|---|---|
| S. No. | Ingredients | weight by weight |
| 1 | Polyethylene oxide | 60.0 |
| 2 | Lactose monohydrate | 38.0 |
| 3 | Magnesium stearate | 1.0 |
| 4 | Colloidal silicon dioxide | 1.0 |

### Drug Layer

| | | Percent (%) |
|---|---|---|
| S. No. | Ingredients | weight by weight |
| 1 | Diclofenac sodium | 5.0 |
| 2 | Lactose monohydrate | 30.0 |
| 3 | Hydroxypropylmethyl cellulose (50 cps to | 30.0 |
| | 10000 cps) | |
| 4 | Methacrylic acid - ethyl acrylate copolymer | 30.0 |
| | (1:1) Type A | |
| 5 | Hydroxypropyl cellulose-L | 4.0 |
| 6 | Isopropyl alcohol (IPA) + water | q.s |
| 7 | Magnesium stearate | 0.5 |
| 8 | Colloidal silicon dioxide | 0.5 |

### Manufacturing Process:

### Famotidine Drug-Layering Dispersion

1. Disperse hydroxypropylmethyl cellulose in purified water.
2. Disperse famotidine in the above hydroxypropylmethyl cellulose dispersion under stirring.

### Polymer Layer

1. Pass polyethylene oxide and lactose monohydrate through a screen and lubricate using magnesium stearate and colloidal silicon dioxide.
2. Compress the blend of step 1 into tablets.

### Drug Layer

1. Accurately weigh ingredients 1-4 of the drug layer and pass through a mesh.
2. Make a binder solution by dissolving hydroxypropyl cellulose-L in the mixture of isopropyl alcohol and water.
3. Granulate the powder mass of step 1 with the binder solution of step 2.
4. Dry the granules of step 3 in a fluidized bed drier.
5. Mill the granules of step 4 and lubricate them using magnesium stearate and colloidal silicon dioxide.
6. Compress the granules of step 5 into tablets.

### Filling of Tablets and Pellets in to a Hydroxypropylmethyl Cellulose Capsule

Fill the tablets in a capsule in the following order:
1. Polymer tablet
2. Polymer tablet
3. Drug core tablet
   Coating of the Filled Hydroxypropylmethyl Cellulose Capsule with Famotidine Dispersion

Load the filled hydroxypropylmethyl cellulose capsules in a conventional pan coating machine and layer the famotidine drug layering dispersion over the capsule to a weight gain equivalent of 5% to 15% weight by weight of famotidine per capsule.

1. An in-situ multilayered tablet comprising at least one polymer layer and at least one drug layer wherein said layers are physically separated from each other.

2. The in-situ multilayered tablet according to claim 1, wherein the said tablet comprises one polymer layer and one drug layer.

3. The in-situ multilayered tablet according to claim 1, wherein the said tablet comprises two polymer layers and one drug layer.

4. The in-situ multilayered tablet according to claim 1, wherein the said tablet comprises two polymer layers and two drug layers.

5. The in-situ multilayered tablet according to claim 1, wherein the polymer layer may optionally comprise a drug.

6. The in-situ multilayered tablet according to claim 1 and claim 5, wherein the polymer layer and the drug layer contain the same drug.

7. The in-situ multilayered tablet according to claim 1 and claim 5, wherein the polymer layer and the drug layer contain different drugs.

8. The in-situ multilayered tablet according to claim 1, wherein the said tablet provides an initial lag phase followed by the controlled release of the drug present in the drug layer wherein the drug layer is sandwiched between the two polymer layers.

9. The in-situ multilayered tablet according to claim 1, wherein the said tablet provides an initial immediate release followed by the controlled release of the drug present in the drug layer, wherein the drug layer contains a polymer layer on either of its sides.

10. The in-situ multilayered tablet according to claim 1, wherein the said tablet comprise an immediate release layer on it.

11. The in-situ multilayered tablet according to claim 10, wherein the said immediate release layer is in the form of a powder or a tablet.

12. The in-situ multilayered tablet according to claim 1, wherein the polymer layer comprises swelling polymers, antiadherents, binders, diluents, disintegrants, glidants, lubricants, opaquants, and/or polishing agents and optionally a drug.

13. The in-situ multilayered tablet according to claim 12, wherein the swelling polymer is selected from the group consisting of polyethylene oxide polymers, polyethylene glycol polymers, hydroxymethylcellulose, hydroxyethylcellulose, hydroxypropyl methylcellulose having molecular weight from 1,000 to 4,000,000, hydroxypropyl cellulose having molecular weight from 2,000 to 2,000,000, carboxyvinyl polymers, polyvinyl alcohols, glucans, scleroglucans, chitosans, mannans, galactomannans, xanthan gum, carrageenan, amylose, alginic acid and salts, and derivatives thereof, polyanhydrides, polyamino acids, methyl vinyl ethers/maleic anhydride copolymers, carboxymethylcellulose and derivatives thereof, acrylates, methacrylates, acrylic/methacrylic copolymers, or mixtures thereof.

14. The in-situ multilayered tablet according to claim 12, wherein the swelling polymer comprises about 50% to about 100% by weight of the polymer layer.

15. The in-situ multilayered tablet according to claim 1, wherein the drug layer comprises a drug and one or more pharmaceutically acceptable excipients selected from the group comprising adsorbents, antioxidants, acidifying agents, alkalizing agents, buffering agents, colorants, flavorants, sweetening agents, antiadherents, binders, diluents, disintegrants, glidants, lubricants, opaquants, and/or polishing agents.

16. The in-situ multilayered tablet according to claim 1, wherein the polymer layer and the drug layer are prepared by the process of direct compression, dry granulation or wet granulation.
